(19) United States
(12) Patent Application Publication
(10) Pub. No.: US 2023/0371896 A1
(43) Pub. Date: Nov. 23, 2023

(54) ELECTROMAGNETIC FIELD CHANNEL FOR PROPAGATING AN ELECTROMAGNETIC FIELD TO A SENSOR WHILE MINIMIZING EXTERNAL INTERFERENCE

(71) Applicants: Dan Miulli, Riverside, CA (US); James Garvin Wiginton, IV, Riverside, CA (US); James Brazdzionis, Redlands, CA (US); Paras Savla, Riverside, CA (US)

(72) Inventors: Dan Miulli, Riverside, CA (US); James Garvin Wiginton, IV, Riverside, CA (US); James Brazdzionis, Redlands, CA (US); Paras Savla, Riverside, CA (US)

(21) Appl. No.: 18/172,676

(22) Filed: Feb. 22, 2023

Related U.S. Application Data
(63) Continuation of application No. 18/149,980, filed on Jan. 4, 2023.
(60) Provisional application No. 63/365,051, filed on May 20, 2022.
(60) Provisional application No. 63/365,049, filed on May 20, 2022.

(51) Int. Cl. A61B 5/00; H05K 9/00; A61B 5/291

(52) U.S. Cl. CPC A61B 5/6803; H05K 9/0049; A61B 5/291; H05K 9/006; A61B 2562/0209; A61B 2562/182; A61B 2562/227

(57) ABSTRACT

A method and device for measuring electromagnetic fields embodied in an electromagnetic field (EMF) channel that defines a lumen for receiving an EMF-measuring sensor. The EMF channel includes coaxial inner first layer of Mu-metal surrounded by a second layer of interlaced mesh copper, wherein the first layer is configured to attenuate the magnetic field and the copper attenuates the electric field of external, undesired EMFs, thereby promote propagation of a desired EMF to the housed senor.

### CROSS-REFERENCE TO RELATED APPLICATION

This application claims the benefit of priority of U.S. provisional application No. 63/365,049 filed 20 May 2022 and claims the benefit of priority of U.S. non-provisional patent application Ser. No. 18/149,980, filed 4 Jan. 2023, as a continuation thereof, the contents of both are herein incorporated by reference.

### BACKGROUND OF THE INVENTION

The present invention relates to methods and devices for measuring electromagnetic fields and more particularly, to an electromagnetic field channel for propagating an electromagnetic field to a sensor while minimizing external interference.

When measuring electromagnetic fields (EMF) using electromagnetic field sensors, the signal being measured decays over distance, limiting the ability to accurately measure the signal from range. For instance, external signals may overpower and mask the desired signal. Thus, to measure a magnetic field from a distance it is necessary to channel that magnetic field while appropriately excluding external signal that may lead to erroneous measurements.

Shielding technologies are geared towards excluding a large area of electromagnetic fields and are typically constructed as a room. Large magnetic shielded rooms have large space requirements as well as large investment costs.

Presently, devices utilized in magnetic shielding to isolate subjects or objects typically also just focus on isolating external electromagnetic fields. As a result, magnetic shielding devices are not specifically designed to isolate shielding to the sensor itself nor allow for the isolated magnetic field of interest to reflect within the channel to better measure said electromagnetic field. In sum, current magnetic shielding devices do not effectively promote propagation of the electromagnetic signal, which results in decay over distance.

A need exists for an electromagnetic field (EMF) channel for propagating an electromagnetic field to a sensor while minimizing external interference, wherein the EMF channel is inexpensive, small, and portable so it can be operatively associated with wearable devices.

### SUMMARY OF THE INVENTION

The present invention embodies a device specifically designed to both shield and propagate electromagnetic fields to a sensor for measurement. This allows for measurements of the electromagnetic field from a variety of distances and allows for integration with additional products utilized for continued shielding. This allows propagation of the electromagnetic signal to extend further than what would be the predicted rate of decay of the signal traveling over a distance (inverse square of the distance).

An EMF channel provides shielding of the external environment through usage of Mu-metal and has an insulating type of effect for the electromagnetic field traveling up its tubular shape allowing it to be measured by the sensor contained in the tube while excluding the external environment. A plastic covering surrounds a layer of copper mesh which has Mu-metal wrapped on the inside. The sensor housing goes inside this channel. The measuring sensor can then be withdrawn or moved closer to the target within the tube to a desired focal length.

Additionally, although Mu-metal has been utilized in a tubular format for shielding, the construction of this Mu-metal tubular design accompanied by an outer layer of interlaced copper mesh with an external plastic tubing just large enough to contain the sensor allows for optimum titration of distance away from a target and is specifically designed to hold sensors.

In one aspect of the present invention, an electromagnetic field (EMF) channel, the EMF channel includes: a tubular lumen defined by a first layer of an alloy configured to attenuate a magnetic field; and a second layer of material directly contacting the first layer, the second layer configured to attenuate an electric field, wherein the first and second layers are coaxial.

In another aspect of the present invention, the EMF channel includes wherein the alloy is Mu-metal, wherein the second layer comprises interlaced copper; further including a third layer directly contacting the second layer, wherein the third layer is coaxial to the second layer, wherein the first layer has a first thickness of approximately 0.014 inches, and wherein the second layer has a second thickness of approximately 0.250 inches.

These and other features, aspects and advantages of the present invention will become better understood with reference to the following drawings, description, and claims.

### BRIEF DESCRIPTION OF THE DRAWINGS

FIG. 1 is a perspective view of an exemplary embodiment of the present invention, shown in use.

FIG. 2 is an exploded perspective view of an exemplary embodiment of the present invention, showing a plug 20 in dashed lines for clarity when viewed in conjunction with FIG. 4.

FIG. 3 is a section view of an exemplary embodiment of the present invention, taken along 3-3 in FIG. 2, illustrating a lumen of the EMF channel 10 without the plug 20.

FIG. 4 is a section view of FIG. 3, illustrating a lumen of the EMF channel 10 with the plug 20, still shown in dashed lines for clarity.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, an embodiment of the present invention provides a method and device for measuring electromagnetic fields embodied in an electromagnetic field (EMF) channel. The EMF channel defines a lumen for receiving an EMF-measuring sensor. The EMF channel includes coaxial inner first layer of Mu-metal surrounded by a second layer of interlaced mesh copper, wherein the first layer is configured to attenuate the magnetic field and the copper attenuates the electric field of external, undesired EMFs, thereby promote propagation of a desired EMF to the housed senor.

It should be understood that the description herein repeatedly refers to Mu-metal. It is noted that the present invention is not particularly limited to Mu-metal. When Mu-metal is discussed, any proper substitute may be utilized. Mu-metal can be any composite that has a nickel-iron soft ferromagnetic alloy with very high permeability, which is used for shielding sensitive electronic equipment against static or low-frequency magnetic fields. It has several compositions. One such composition is approximately 77% nickel, 16% iron, 5% copper, and 2% chromium or molybdenum. Mu-metal is considered to be ASTM A753 Alloy 4 and may be composed of approximately 80% nickel, 5% molybdenum, small amounts of various other elements such as silicon, and the remaining 12 to 15% iron. In another embodiment, Mu-metal is any soft metal configured or selected to block an electromagnetic field.

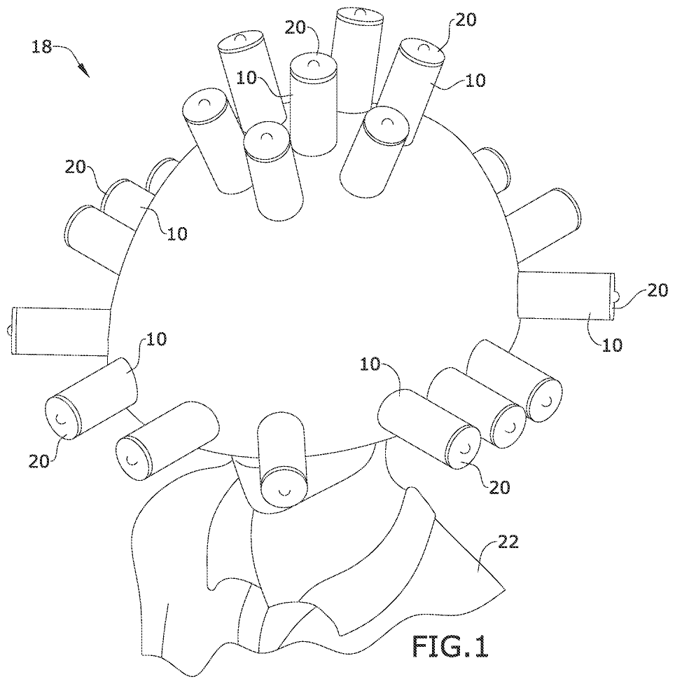

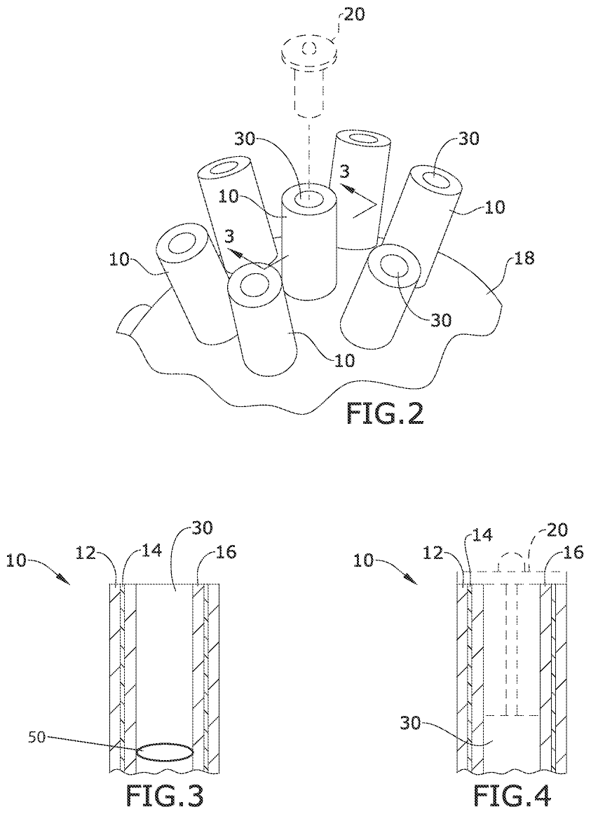

Referring to FIGS. 1-4, the present invention may include a method and device for measuring electromagnetic fields embodied in an electromagnetic field (EMF) channel 10. The EMF channel 10 may be tubular and dimensioned to receive a sensor configure to measure electromagnetic fields. These sensors are reliant on shielding to exclude external electromagnetic fields. The sensors can be placed within the EMF channel at a desired distance away from a target, such as the human brain.

EMF channel 10 may include three coaxial layers. An outer layer 12, an intermediate layer 14, and an inner layer 16. The inner layer 16 may be Mu-metal sized such that the sensor 50 fits snugly within the lumen defined thereby. The Mu-metal layer 16 provides attenuation of the magnetic field. The intermediate copper mesh layer 14 surrounds the Mu-metal layer 16, and the copper attenuates the electric field. This Mu-metal layer 16 and interlaced copper mesh layer 14 allows for the electromagnetic field that enters the opening 30 of the channel 10 to reflect within the lumen of the EMF channel 10 and travel towards the sensor to reduce decay and external interference, enabling improved measurements of EMFs by the sensor(s). One or more EMF channels 10 can be connected to other shielding devices 18, such as helmets, that may contain the subject or object of interest.

The inner layer of Mu-metal layer 16 provides the appropriate shielding to exclude external electromagnetic fields and propagates the electromagnetic field of interest at the end of the tube allowing it to reflect within the tubular shape and be measured by the sensor within the electromagnetic field channel. The Mu-metal layer 16 provides propagation of the internal magnetic field and attenuation of the external magnetic field.

The designated length of the electromagnetic field channel 10 would be selected based on a desired maximal measurement distance. In the context wherein the target is a human brain, the desired maximal measurement distance depends on the distance from the subject (human brain) and that distance can be modulated depending on what is being measured or the depth of measurement. A sheet of Mu-metal would be cut the length of the desired channel and then formed into a tube to form the internal layer 16 of Mu-metal. In one embodiment the Mu-metal layer 16 has a thickness of approximately 0.014 inches and is wrapped inside a layer of interlaced copper mesh (layer 14). The copper mesh layer 14 may, in certain embodiments, measure approximately 0.25 inches and could be wrapped around the Mu-metal layer 16 for the length of the electromagnetic field channel 10. The length of the channel is variable based on the size of the sensor that will be used.

The outer layer 12 is plastic and provides support and would wrap around the interlaced copper mesh layer 14. This entire construct could be secured to another shielding device 18 as needed using adhesive or welding materials to other layers of Mu-metal. The hole 30 formed by the inner Mu-metal layer would be the diameter of the desired sensor housing utilized. A sensor could then be placed in the housing and moved up and down the channel to a desired distance.

The internal layer 16 of Mu-metal provides the appropriate shielding to exclude external electromagnetic fields and propagates the electromagnetic field of interest at the end of the tube allowing it to reflect within the tubular shape and be measured by the sensor within the EMF channel 10. In other words, the internal magnetic field of interest is reflected and channeled up to the EMF channel whereas the external electromagnetic fields have no way of being channeled and are thereby reflected away.

The layer of interlaced copper mesh 14 is also critical regarding mitigating external signals and possibly propagating the electromagnetic field of interest by working in conjunction with the internal layer of Mu-metal 16 as the he copper mesh layer is responsible for electric field mitigation whereas the mu-metal layer is responsible for magnetic field mitigation. Thus, the copper mesh provides attenuation of the electric field. The outer plastic layer 12 is necessary to provide the structure for the copper and Mu-metal layers 14 and 16, but the specifics of plastic type and thickness are not critical to the function.

This device can be utilized to measure electromagnetic fields from a distance by shielding the sensor from external, undesired external electromagnetic field and simultaneously propagating the electromagnetic field of interest from the end of the EMF channel. This design additionally limits the amount of shielding needed and utilizes fewer materials and space than a full shielded room which is commonly utilized in these experiments and for these purposes. The design allows for the sensor distance to be manipulated as needed by the user to withdraw and advance the sensor housing through the channel away from the subject of interest, in some embodiments the subject of interest is the wearer of the helmet.

On application of the present invention would be for an electromagnetic field helmet 18 for a wearer 22, which has a plurality of EMF channels 10 radially extending from an outer surface of the helmet 18. A plug 20 may be provided for each EMF channel 10, wherein the plug 20 slides into the hole 30 of the EMF channel 10.

As used in this application, the term “about” or “approximately” refers to a range of values within plus or minus 10% of the specified number. And the term “substantially” refers to up to 80% or more of an entirety. Recitation of ranges of values herein are not intended to be limiting, referring instead individually to any and all values falling within the range, unless otherwise indicated, and each separate value within such a range is incorporated into the specification as if it were individually recited herein.

For purposes of this disclosure, the term “aligned” means parallel, substantially parallel, or forming an angle of less than 35.0 degrees. For purposes of this disclosure, the term “transverse” means perpendicular, substantially perpendicular, or forming an angle between 55.0 and 125.0 degrees. Also, for purposes of this disclosure, the term “length” means the longest dimension of an object. Also, for purposes of this disclosure, the term “width” means the dimension of an object from side to side. For the purposes of this disclosure, the term “above” generally means superjacent, substantially superjacent, or higher than another object although not directly overlying the object. Further, for purposes of this disclosure, the term “mechanical communication” generally refers to components being in direct physical contact with each other or being in indirect physical contact with each other where movement of one component affect the position of the other.

The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

In the following description, it is understood that terms such as “first,” “second,” “top,” “bottom,” “up,” “down,” and the like, are words of convenience and are not to be construed as limiting terms unless specifically stated to the contrary.

It should be understood, of course, that the foregoing relates to exemplary embodiments of the invention and that modifications may be made without departing from the spirit and scope of the present invention.

What is claimed is:

1. An electromagnetic field (EMF) channel, the EMF channel comprising:
a tubular lumen defined by a first layer of an alloy configured to attenuate a magnetic field; and
a second layer of material directly contacting the first layer, the second layer configured to attenuate an electric field, wherein the first and second layers are coaxial.

2. The EMF channel of claim 1, wherein the alloy is Mu-metal.

3. The EMF channel of claim 2, wherein the second layer comprises interlaced copper.

4. The EMF channel of claim 3, further comprising a third layer directly contacting the second layer, wherein the third layer is coaxial to the second layer.

5. The EMF channel of claim 4, wherein the first layer has a first thickness of approximately 0.014 inches.

6. The EMF channel of claim 5, wherein the second layer has a second thickness of approximately 0.250 inches.

7. The EMF channel of claim 6, an EMF sensor housed in the lumen.

8. The EMF channel of claim 1, wherein the alloy comprises a soft metal selected to block an electromagnetic field.
